(19) United States
(12) Patent Application Publication
(10) Pub. No.: US 2012/0277518 A1
(43) Pub. Date: Nov. 1, 2012

(54) Brachytherapy Device and Method

(76) Inventors: Felix W. MICK, Bronxville, NY (US); Paul DASILVA, Flushing, NY (US)

(21) Appl. No.: 13/458,713

(22) Filed: Apr. 27, 2012

Related U.S. Application Data
(60) Provisional application No. 61/479,839, filed on Apr. 27, 2011.

(51) Int. Cl. A61M 36/10

(52) U.S. Cl. 600/6

(57) ABSTRACT

A device and method for retaining a position of a treatment applicator include a perineal retainer body configured to receive the applicator and form a positive stop with a perineum of a body of a patient, and a locking bracket configured to secure the perineal retainer body to the applicator. The locking bracket is movable between first and second positions with respect to the perineal retainer body, is configured to exert a clamping force to clamp the applicator, includes an actuator configured to move the locking bracket between the first and second positions, and is vertically accessible for securing and adjusting the applicator with respect to the perineal retainer body. The method includes securing a perineal retainer body to the applicator by a locking bracket, and attaching the perineal retainer body to a body of a patient.

### CROSS-REFERENCE TO RELATED APPLICATION

The present application claims the benefit of U.S. Provisional Patent Application No. 61/479,839, filed on Apr. 27, 2011, which is expressly incorporated herein in its entirety by reference thereto.

### FIELD OF THE INVENTION

The present invention relates generally to a brachytherapy device and to a method for cervical brachytherapy treatment using a brachytherapy device. Radioactive therapeutics may be delivered to a region of the cervix, e.g., to provide for treatment of cervical cancerous tissue.

### BACKGROUND INFORMATION

Brachytherapy is a form of cancer treatment in which radiation sources are placed inside a patient's body, e.g., to irradiate a tumor. In brachytherapy, a physician typically implants several radioactive seeds in or around a tumor, thereby providing a higher radiation dose to the tumor than would be possible with external beam radiation therapy. Careful placement of the radioactive seeds is critical to allow for localized and precise irradiation of the tumor.

Cervix applicators using radiation in the treatment of cervical cancer have been in existence since the discovery of Radium in the early 19th century. Historically, Radium sources (sealed sources) were placed in ovoids attached to tubes and thereafter inserted “manually” into vaginal or intrauterine cavities for the treatment of cancer. The insertion of the applicator was associated with significant radiation exposure to the physicians handling the apparatus.

Henschke et al. at Memorial Hospital in New York are believed to have introduced the “Afterloading Technique” in 1960, in which applicators are inserted first and the radioactive materials thereafter. Other isotopes, such as cesium-137 and iridium-192 sources have slowly replaced Radium. Cervix applicators have been developed in many different versions, and one widely accepted today is the Fletcher-Suit-Delclos (FSD) applicator. This applicator consists of two lateral ovoids and one central tandem. The ovoids can be spread laterally and fixed in place. The tandem is floating, and packing is required. Applicators in use today are considered “manual afterloading applicators.”

In the early 1980s, High-Dose-Rate (HDR) Remote Afterloading is believed to have been introduced in the United States. These units provided greater protection from radiation exposure to staff and personnel. Applicators were inserted “cold” in specially shielded rooms, and the radioactive sources were inserted “remotely” from control consoles located outside the treatment room. The HDR break-through featured sealed sources much smaller in size than radium or cesium sources and opened new treatment possibilities and with it new opportunities in applicator designs.

A potential difficulty that remains with cervical brachytherapy applicators is that a GYN applicator inserted into the uterus or the vaginal cavity may have a tendency to dislocate, e.g., by internal muscular pressure which could result in serious consequences and misadministrations during high-dose-rate (HDR) irradiation.

Thus, it is desirable to provide a method to retain the position of the brachytherapy applicator during irradiation.

Further it is desirable to provide a device for retaining the position of the applicator that is ergonomic, easy to operate, effective, and easily cleaned and sanitized.

### SUMMARY

In accordance with example embodiments of the present invention, a method to retain the position of an applicator during irradiation is accomplished by attaching a perineal bar or retainer body to the applicator, wherein the perineal bar is then held in place via straps looped through, e.g., four access holes and around the patient's torso. Example embodiments also include a vertically accessible locking and adjustment mechanism for locking and/or adjusting the position of the applicator with respect to the perineal bar or retainer body.

A vertically accessible locking/adjusting mechanism is advantageous in comparison to side-locking mechanisms. For example, heavy patients with heavy thighs may be problematic when trying to manipulate screws or knobs laterally. The thighs may get in the way and obstruct access to the locking screws. Thus the vertical locking mechanism provides desirable benefits with regard to accessibility and ergonomics.

In accordance with example embodiments of the present invention, the vertical locking screw when tightened, lifts a tapered fork (resembling a tuning fork) and simultaneously squeezes a polymeric component together which then clamp onto, e.g., two parallel tubes of the applicator, which may correspond to, e.g., a tandem and a ring tube.

The locking screw may be rotatable about an axis that is at least one of (a) substantially coplanar with the longitudinal axes of the two parallel tubes, (b) substantially parallel to the plane containing the longitudinal axes of the two parallel tubes, and/or (c) substantially perpendicular to the longitudinal axes of the two parallel tubes.

In accordance with example embodiments of the present invention, a device for retaining a brachytherapy applicator includes a perineal retainer body configured to receive and clamp the applicator, the retainer body being configured to form a positive stop with the perineum of a patient when the applicator is clamped by the retainer body and inserted into the patient, a locking bracket movable between a first position and a second position with respect to the retainer body, the locking bracket configured to exert a clamping force to clamp the applicator when the locking bracket moves from the first position to the second position, and an actuator configured to move the locking bracket between the first position and the second position.

The movement of the locking bracket between the first and second positions may be along a line that substantially falls in a plane containing the anteroposterior axis and the dorsoventral axis of the patient when the clamped applicator is inserted into the patient.

The actuator may be manually accessible in a direction substantially falling along the plane containing the anteroposterior axis and the dorsoventral axis of the patient when the clamped applicator is inserted into the patient.

The actuator may be a lead screw forming a threaded connection with the locking bracket.

The locking bracket may have a camming surface configured to slide across a respective camming surface of the retainer body as the locking bracket is moved from the first position to the second position.

The device may be configured to clamp two parallel tubes of the applicator, which may correspond to, e.g., a tandem and a ring tube.

The locking bracket may be configured to move with respect to the retainer body along an axis that is at least one of (a) substantially coplanar with the longitudinal axes of the two parallel tubes, (b) substantially parallel to the plane containing the longitudinal axes of the two parallel tubes, and/or (c) substantially perpendicular to the longitudinal axes of the two parallel tubes.

Example embodiments of the present invention are described in more detail below with reference to the appended Figures.

### BRIEF DESCRIPTION OF THE DRAWINGS

FIGS. 1 and 2 are perspective views of a perineal retainer attached to a CT compatible HDR ring and tandem applicator.

FIG. 3 shows the perineal retainer of FIG. 1 in an open state.

FIG. 4 shows the perineal retainer of FIG. 1 with the locking bracket and locking screw removed from the retainer body.

FIGS. 5 and 6 show the perineal retainer of FIG. 1 in open and closed states, respectively.

FIG. 7 shows a lock screw tool configured to convert the perineal retainer of FIG. 1 between the open state and the closed state.

FIGS. 8 and 9 sequentially illustrate the assembly of the perineal retainer of FIG. 1.

FIGS. 10 and 11 sequentially illustrate the attachment and securement of the perineal retainer of FIG. 1 to the HDR ring and tandem applicator of FIG. 1.

### DETAILED DESCRIPTION

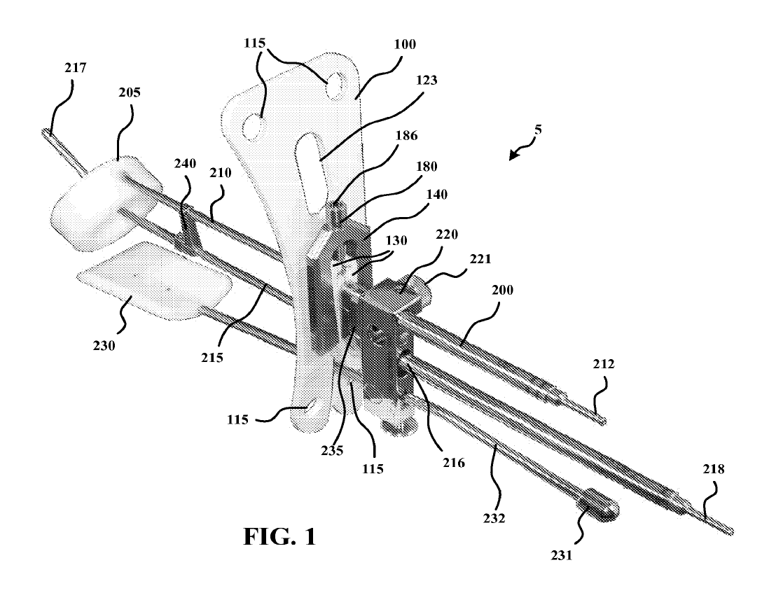

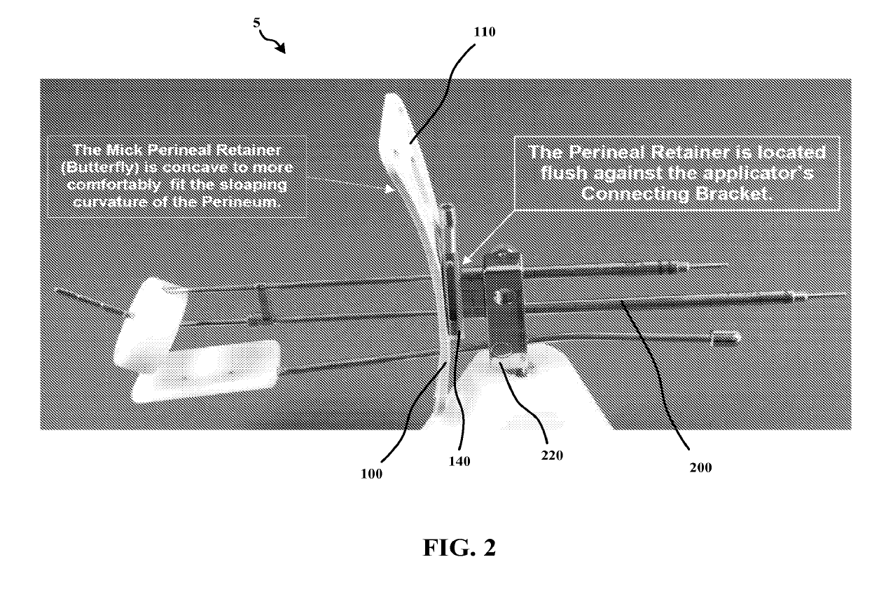

FIGS. 1 and 2 are perspective views of an assembly 5 of a perineal retainer 100 attached to a CT compatible high dosage radiation (HDR) ring and tandem applicator 200 according to an example embodiment of the present invention. Although a specific applicator 200 is described in connection with the illustrated embodiment, it should be understood that any suitable applicator may be provided. For example, any applicator described in U.S. Pat. No. 7,666,130, which is hereby incorporated by reference in its entirety, may be provided for the applicator 200.

It noted that FIG. 2 shows the assembly 5 supported on a support cone for purposes of illustration. However, the cone does not form part of the assembly 5.

The perineal retainer 100, which is described in greater detail below, is provided as an accessory to an HDR ring and tandem applicator 200, such as the Mick CT Compatible HDR Ring and Tandem Applicator (e.g., the 32 mm set) commercially available from Mick Radio-Nuclear Instruments, Inc. of Mt. Vernon, N.Y. The general purpose of the perineal retainer is to retain the ring and tandem applicator in the patient to the required depth and to avoid applicator slippage in the patient. It is generally intended for use where high dose rate (HDR) radiation is an accepted clinical practice in the treatment of the cervix and the urethra.

The perineal retainer 100 may be reusable in its entirety and may be properly cleaned and sterilized prior to every use. For example, the perineal retainer 100 may be autoclaved repeatedly without any substantial alteration and/or damage to the physical structure and mechanical properties of perineal retainer 100. It should be understood, however, that retainer may be, in whole or in part, disposable.

The perineal retainer 100 includes a body (or “Butterfly”) 110 that may be affixed to the patient's body by any appropriate mechanism, e.g., suturing, strapping, and/or any other preferred methods or mechanism. Such securement of the perineal retainer 100 may be advantageous to prevent slippage of the applicator 200, which is supported by the perineal retainer 100, with respect to the patient's body from the predetermined applicator position. When affixed, the perineal retainer 100 according to the example embodiment contacts the perineum of the patient to basically form a positive stop to prevent over-insertion of the applicator 200 beyond its predetermined position in the patient's body. Similarly, the securement of the perineal retainer 100 to the patient's body (e.g., via straps, etc.) prevents unintended retraction of the applicator 200 from its predetermined position in the patient's body. That is, the securement holds the perineal retainer 100 against the patient's body.

To facilitate strapping of the perineal retainer 100 to the patient's body, the perineal retainer body 110 includes strapping attachment provisions in the form of four apertures 115 configured to receive respective end loops of one or more strap members configured to, e.g., extend around the patient's body.

The HDR ring and tandem applicator 200, which is supported by the perineal retainer 100, includes ring 205, ring support tube 210, central tandem 215, and universal connector 220. The ring applicator 200 is designed for insertion into a cervix, generally after the cervix has been dilated through the use of an appropriate pharmaceutical. After ring applicator 200 has been inserted into the cervix, radiation is provided, e.g., from iridium-192, at ring 205.

The universal connector 220 (a coupling device) connects ring support tube 210 and central tandem 215. The distal end of ring support tube 210 is connected to ring 205 to support ring 205 in a predetermined location relative to ring support tube 210. Universal connector 220 includes lock nut 221 so that the relative positioning of the ring 205 and tandem 215 may be provided and secured. Central tandem locator 216 is formed as a part of central tandem 215 and connects to universal connector 220. Central tandem locator 216 positions central tandem 215 such that central tandem end section 217 is central (or concentric) to ring 205. Central tandem 215 may be, for example, rigidly attached to universal connector 220 such that central tandem 215 does not “float.” A radiation dose is deliverable, e.g., via an HDR after loader, to a cervical wall by the HDR ring 205.

Support brackets 235 and 240 are provided to maintain the relative positioning between the central tandem end section 217 and HDR ring 205 by forming rigid supports between the ring support tube 210 and the central tandem 215.

Interface connector 212 and tandem interface connector 218 provide for connecting applicator 200 to, e.g., an HDR afterloader. Interface connector 212 and tandem interface connector 218 may be compatible with all HDR afterloaders. The connectors 212, 218 may be identical, or each connector 212, 218 may be different so that the connectors 212, 218 are connectable only to a corresponding connector of the afterloader, e.g., to avoid misconnections to the afterloader.

Although ring 205 is provided as a single monolithic portion, the ring 205 may be provided as any number of split portions. For example, adjustable split ring tubes, such as provided in U.S. Pat. No. 7,666,130, which is incorporated herein its entirety by reference thereto, may be provided. Further, the portion 205 may form different shapes than a ring.

The applicator 200 also includes a rectal retractor 230. Rectal retractor 230 is attached to the remainder of the applicator 200 through universal connector 220. Rectal retractor 230 is inserted vaginally. Rectal retractor 230 may be adjusted to exert a force on a rectum and may be locked in place. The adjustment may be made via handle portion 231 of retractor shaft 232 of the retractor 230. The shaft 232 is also supported by the universal connector 220.

In order to support the applicator 200 in its fixed position relative to the patient's cervix, the perineal retainer 100 includes a slot 120 formed between two adjacent wings 125 of the perineal retainer body 110. The slot 120 includes a clamping region 121 configured to receive and clamp ring support tube 210 and central tandem 215, thereby securing the applicator 200 in its predetermined position when the perineal retainer 100 is secured to the patient's body.

The slot 120 also includes an enlarged region 122 below the clamping region, which extends to the open lower end of the slot 120. The enlarged region 122 of the slot 120 is configured to (a) allow ease of passage of the ring support tube 210 and the central tandem 215 during assembly, since the enlarged portion 122 is dimensioned to allow clearance and/or reduced friction in comparison to the clamping portion 121 when the perineal retainer 100 is in an open (or unclamped) state, and (b) allow movement of the rectal retractor shaft 232 along the slot even when the perineal retainer 100 is in a closed (or clamped) state, thereby allowing the operator to manipulate the rectal retractor 230 relative to the ring 205 and tandem 215 when the applicator 200 and perineal retainer 100 are secured in the predetermined position relative to the patient.

In order to secure the ring support tube 210 and the central tandem 215 relative to the perineal retainer body 110, the perineal retainer 100 is adjusted from the open state to the closed state. In the illustrated example, the mechanism for doing so involves an interface between the perineal retainer body 110, locking bracket 140, and locking screw 180.

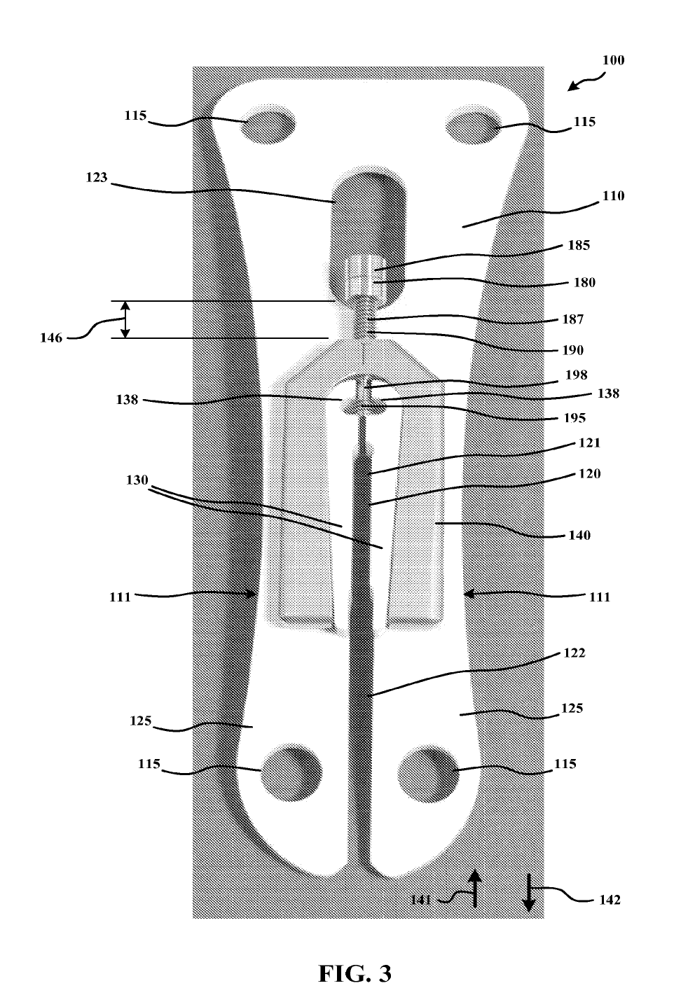

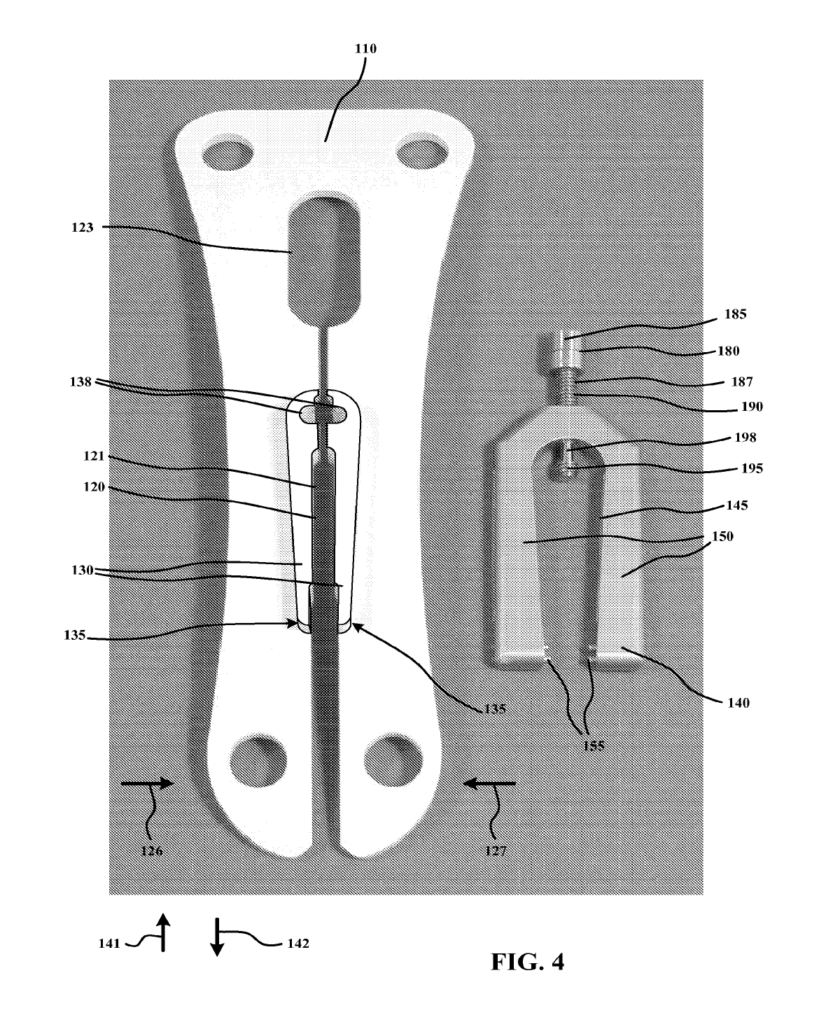

Referring, e.g., to FIGS. 3 and 4, the locking bracket 140 is rigid and generally shaped as an inverted (or upside-down) U-shaped member having a camming channel 145 formed between two opposed camming projections 150, which form a raised portion of the perineal retainer body 110. When the perineal retainer 100 is assembled, the camming channel 145 receives projections 130, each of which extends transversely from the downward extension of a respective wing 125. Thus, the projections 130 extend outwardly from wings 125 in a direction generally aligned with the direction of insertion of the applicator 200 into the patient.

The locking screw 180 includes a head portion 185 connected to a shaft having a threaded locking bracket engagement portion 190 and a perineal retainer body engagement portion 195. When the perineal retainer 100 is assembled, the external threads of the locking bracket engagement portion 190 engage corresponding internal threads of a bore of the locking bracket 140 through which the shaft 187 extends. Further, the perineal retainer body engagement portion 195, which has an enlarged diameter in comparison to the reduced diameter portion 198, is received and axially constrained, but allowed to rotate, by a pair of opposed slots or grooves 138 in respective projections 130. Further, in the assembled state of the perineal retainer 100, the locking bracket 140 is slidable with respect to the perineal retainer body 110 generally along the axis of the locking screw 180. To secure the locking bracket 140 while allowing slidability, the locking bracket 140 includes two opposed guide elements, each in the form of a pair of mounting pins 155, which may be provided, e.g., by pressing the pins 155 into corresponding bores in the camming projections 150.

Since the locking bracket 140 is prevented from rotating about the axis of the locking screw 180, rotation of the locking screw 180 about its axis with respect to the perineal retainer body 110 also results in a corresponding rotation of the locking screw 180 about its axis with respect to the locking bracket 140. Thus, rotation of the locking screw causes a linear translation of the locking screw 180 and the locking bracket 140 with respect to each other due to the threaded engagement between the threaded portion 190 of the shaft 187 of the locking screw 180 and the internal threads of the corresponding bore in the perineal retainer body 110. Further, since the enlarged perineal retainer body engagement portion 195 is axially constrained in notches or slots 138 of the perineal retainer body 110, the relative translation between the locking bracket 140 and the locking screw 180 results in a corresponding translation between locking bracket 140 and the perineal retainer body 110.

Thus, in the assembled state of the perineal retainer 100, rotation of the locking screw 180 in a first rotational direction, e.g., clockwise, causes translation of the locking bracket 140 in a first direction 141, and rotation of the locking screw in a second, opposite rotational direction, e.g., counter-clockwise, causes translation of the locking bracket 140 in a second direction 142 opposite the first direction 141.

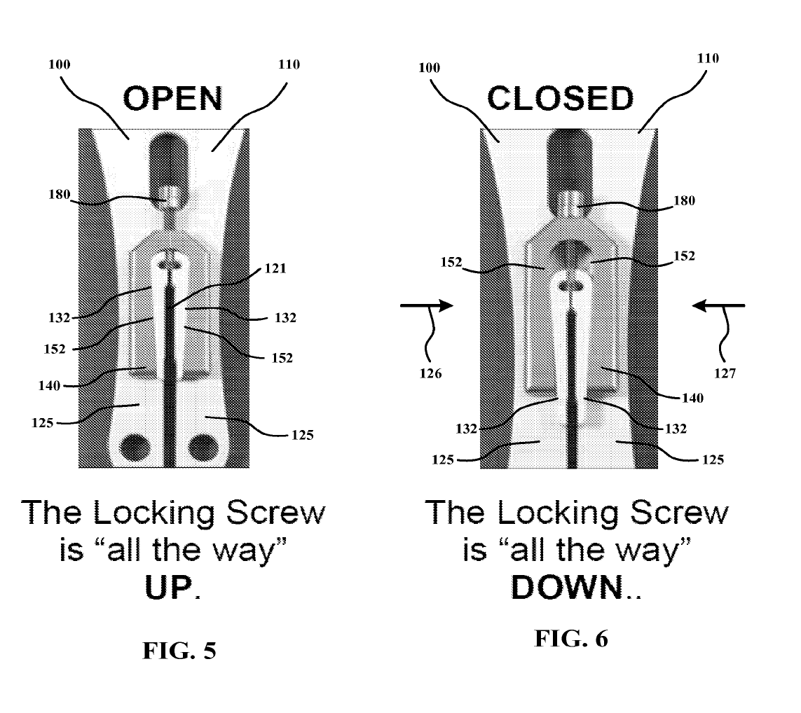

FIGS. 3 and 5 show the perineal retainer 100 in its open state, with the locking bracket 140 in its lowermost position relative to the perineal retainer body 110. Thus, relative to the locking bracket 140, the locking screw 180 is in its uppermost position.

FIG. 6 shows the perineal retainer 100 in its closed state, with the locking bracket 140 in its uppermost position relative to the to the perineal retainer body 110. Thus, relative to the locking bracket 140, the locking screw 180 is in its lowermost position.

Accordingly, when the locking screw 180 is rotated about its axis in the first rotational direction, the perineal retainer 100 may be transitioned from its open state to its closed state. Likewise, when the locking screw 180 is rotated about its axis in the second rotational direction, the perineal retainer 100 may be transitioned from its closed state to its open state.

Movement of the locking bracket 140 in the first direction 141 causes camming surfaces 152 of the locking bracket 140 to translate in the first direction 141 with respect to the respective camming surfaces 132 of the projections 130 of the perineal retainer body 110. The camming surfaces 152 of locking bracket 140 are in contact with the camming surfaces 132 of the perineal retainer body 110 during the translation of the locking bracket 140 relative to the perineal retainer body 110. Further, the camming channel 145 is tapered such that the trailing portions of the camming surfaces 152 are closer together than the leading portions of the camming surfaces 152 during movement of the locking bracket 140 in the first direction. Thus, movement of the locking bracket 140 in the first direction from the open position to the closed position causes the camming surfaces 132 of the of the perineal retainer body 110 to be pressed inwardly toward each other as the camming surfaces 152 slide therealong. In this regard, the camming surfaces 152 act as cams, and camming surfaces 132 act as cam followers in response to movement of the camming surfaces 152.

This inwardly directed force, in directions 126, 127, causes the clamping portion 121 of the slot 120 of the perineal retainer body 110 to narrow as the locking bracket is translated in the first direction with respect to the perineal retainer body 110. Thus, when the perineal retainer is in the open state, the ring support tube 210 and the central tandem 215 are relatively easily slidable into their predetermined positions in the clamping portion 121 of the slot 120. Once the ring support tube 210 and the central tandem 215 are in their predetermined positions, the locking screw 180 may be rotated in the first rotational direction to cause the clamping portion 121 to narrow and constrict to apply a laterally directed clamping pressure on the ring support tube 210 and the central tandem 215, thereby locking or securing the ring support tube 210 and the central tandem 215 in their predetermined positions relative to the perineal retainer 100.

Similarly, rotation of the locking screw in the second rotational direction allows the clamping portion 121 of the slot 120 to expand, thereby removing the clamping force in order to release the ring support tube 210 and the central tandem 215 from their locked or secured position. As indicated above, the camming channel 145 is tapered. Thus, when the locking bracket 140 is moved in the second direction 142 from its closed position to its open position, the trailing portions of the camming surfaces 152 are further apart than the leading portions of the camming surfaces 152. Thus, movement of the locking bracket 140 in the second direction 142 from the open position to the closed position allows the camming surfaces 132 of the perineal retainer body 110 to expand outwardly away from each other as the camming surfaces 152 slide therealong. Contact between the camming surfaces 132 and 152 may be maintained during this expansion of the clamping portion 121 due to e.g., internal spring force in the wings 125 and/or outward force exerted by the ring support tube 210 and/or central tandem 215.

Because the areas of contact between the camming surfaces 132 and the camming surfaces 152 expands along the entire length of the clamping portion 121, an even clamping pressure and force distribution is provided along the length of the clamping portion 121 of slot 120. Thus, it is ensured that both the ring support tube 210 and the central tandem 215 are securely clamped in their predetermined positions.

Further facilitating the evenness of the clamping pressure and force distribution along the length of the clamping portion 121 is that the wings 125 are able to move in a parallel manner (or without substantial rotation) in the region of the clamping region 121 during the contraction of the clamping portion. To facilitate this feature, the slot 120 of perineal retainer 100 extends into an enlarged cutout 123 configured to increase the flexibility of the wings 125 by reducing the material structure in the upper portion of the perineal retainer body 110 above the wings 125.

Although the illustrated example shows the clamping of two structures 210, 215, it should be understood that any number of structures, including a single structure or more than two structures may be clamped in the clamping portion 121. Further, although the clamping portion 121 is a single continuous portion, it should be understood that the clamping portion 121 may be provided at multiple separate locations along the slot 120.

Although the locking bracket 140, which is made of a strong material, e.g., titanium, is sufficiently rigid to maintain the inward clamping force on the perineal retainer body 110 despite the open U-shaped configuration, it should be understood that other configurations may be provided. For example, the locking bracket 140 may be formed in a non-U-shaped structure or a support member may be provided, e.g., near the open end of the U-shaped structure.

Referring, e.g., to FIG. 3, the range of movement 146 of the locking bracket 140 relative to the perineal retainer body 110 is bounded by a positive stop formed between the upper portion of the camming channel 145 of the locking bracket 140 and the upper portion of the projections 130 of the perineal retainer body 110. It should be understood, however, that the limits of the movement of the locking bracket 140 relative to the perineal retainer body 110 may be provided by any other suitable mechanism(s). Further, during use, the upper translation limit of the locking bracket 140 may be determined by the positive stop mentioned above and/or frictional resistance due to increased clamping force between the camming surfaces 132, 152.

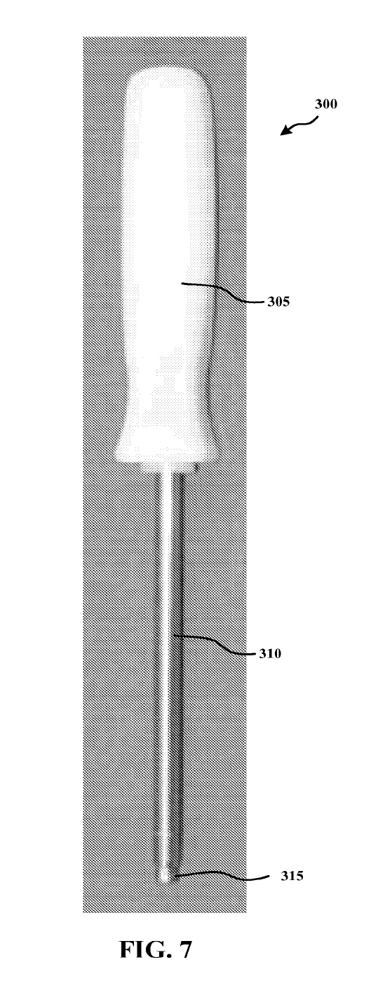

Rotation of the locking screw 180 in the first and second rotational directions may be performed using a lock screw tool 300, illustrated, e.g., in FIG. 7. The lock screw tool 300 includes a handle 305 formed of, e.g., an autoclavable material, and a driving shaft 310 formed of, e.g., stainless steel(s), which is also autoclavable. Thus, the lock screw tool 300 is, as an entire unit, autoclavable. The driving shaft 310 extends from the handle to the driving interface or connector 315 that is keyed (e.g., as a hexagonal or other keyed structure) to a driving receptacle 186 in the head portion 185 of the locking screw 180, which is configured to receive the driving connector 315 in order to allow rotation of the locking screw 180 via rotation of the handle 305 of the lock screw tool 300. It should be understood, however, that any suitable mechanism for rotating the locking screw 180 may be provided. For example, a ratcheting driving mechanism and/or a manual thumb screw may be provided.

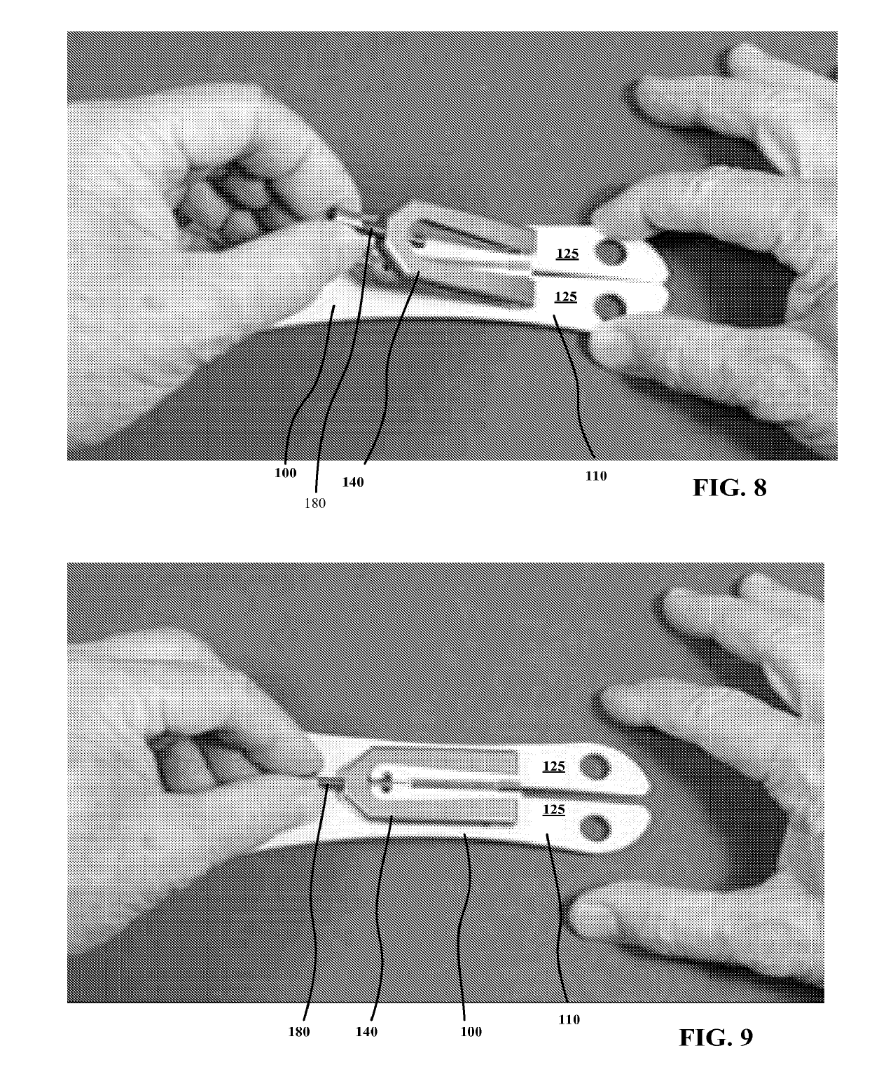

FIGS. 8 and 9 sequentially illustrate the assembly of the perineal retainer 100. As illustrated in FIG. 8, the locking bracket 140 is mounted onto the perineal retainer body 110 by inserting the mounting pins 155 at the bottom of the locking bracket 140 into grooves 135 of the raised section, corresponding to projections 130, of the perineal retainer body 110. This is performed easily by pinching together, e.g., manually, the two wings 125 at the bottom of the perineal retainer body 110. The user may then ensure that the locking screw 180 is fully “extended” such that the locking screw 180 is in its uppermost position relative to the locking bracket 140 such as when the locking bracket 140 is in its fully open position in the assembled perineal retainer 100.

Referring to FIG. 9, to complete the assembly, the user ensures that the short threaded section 195, which is the perineal retainer body engagement portion 195, of the locking screw 180 is inserted in the oval-shaped notch 138 in the top of the raised section 130, corresponding to projections 130, of the perineal retainer body 110. The user may then again ensure that the locking screw 180 is still “fully” extended, thus ensuring that the assembled perineal retainer 100 is in its open state.

It is noted that the perineal retainer body engagement section 195 of the locking screw 180 is threaded in order to allow the section 195, which is enlarged with respect to reduced diameter portion 198 of the shaft 187, to be passed through the internally threaded bore of the locking bracket 140 by rotating the threaded connection until the section 195 disengages the internal threads of the bore. Further axial extension then allows engagement of the threads of the threaded locking bracket engagement portion 190 to engage the internal threads of the bore. This arrangement may facilitate manufacture of the locking screw, since a continuously threaded shaft may be machined, e.g., on a lathe, to form the reduced diameter portion 198, to thereby also delineate the portions 190 and 195 adjacent the reduced diameter portion 198. It should be understood, however, that any suitable manufacturing process may be provided and that the perineal retainer body engagement portion 195 may be provided without threads and/or be of a different diameter than the threaded locking bracket engagement portion 190.

It is further noted that, while the lower portion the locking bracket 140 is maintained secured by the engagement of mounting pins 155 in grooves 135, the upper portion is secured in its installed position via the engagement of the reduced diameter portion 198 of the locking screw 180 with the perineal retainer body portion 195. In particular, the reduced diameter portion 198 is pressed between two opposed grooves (extending in the general direction of the longitudinal axis of the locking screw 180) in the perineal retainer body portion 195, whereby the grooves partially encircle the reduced diameter portion 198 such that the reduced diameter is pushed and snapped into place between the grooves in the manner of a detent. If should be understood, however, than any suitable securement mechanism may be provided.

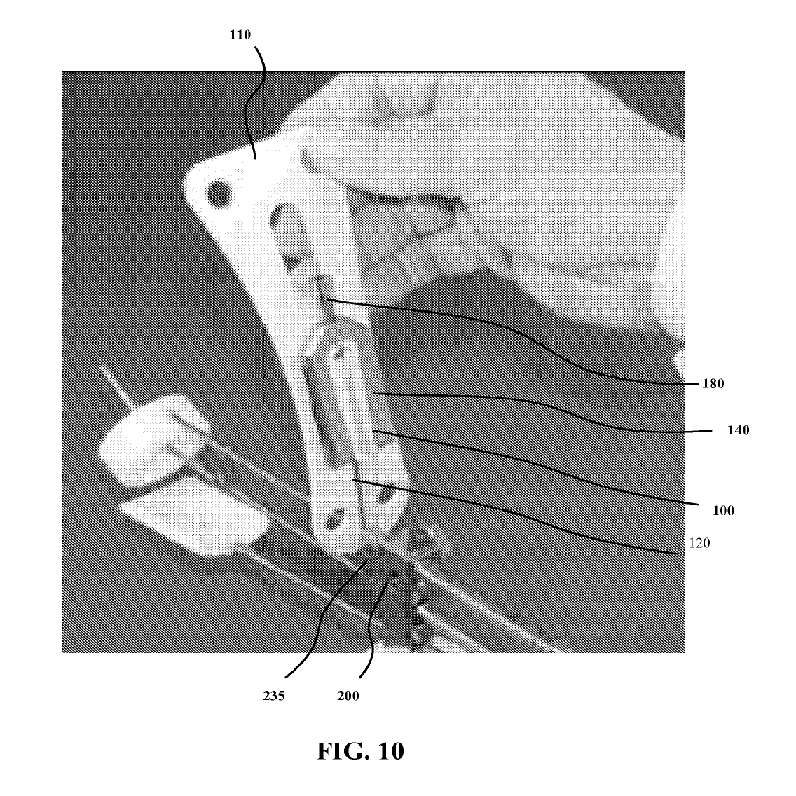

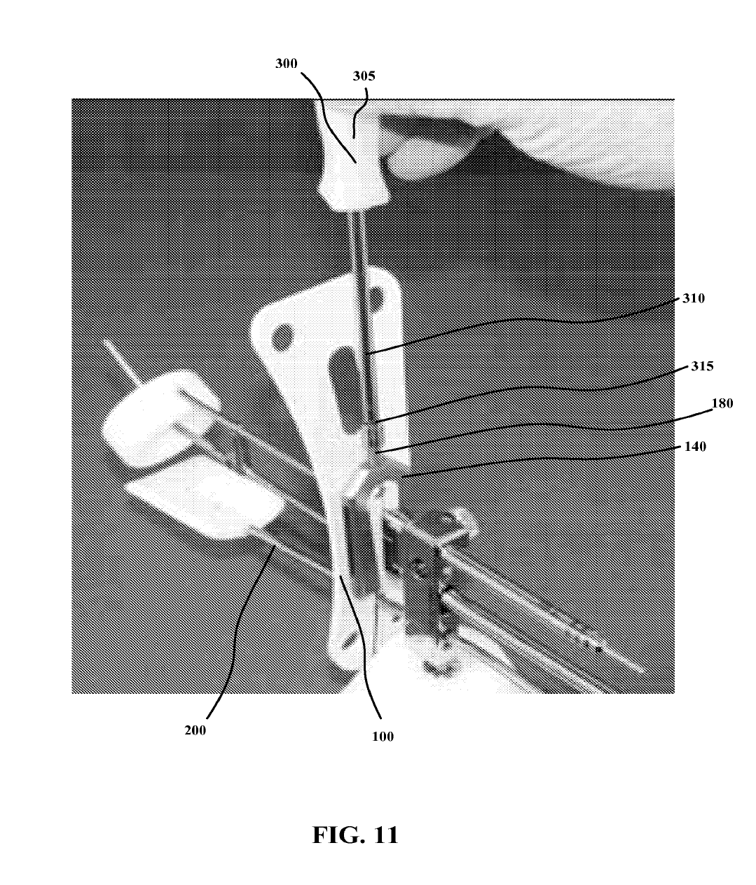

FIGS. 10 and 11 sequentially illustrate, respectively, the attachment and securement of the perineal retainer 100 to the HDR ring and tandem applicator 200. Referring to FIG. 10, the user slides the assembled perineal retainer 100 in its open state, as shown, vertically down over the tandem 215 and ring tube 210 as close as possible to the ring and tandem connecting bracket 235. The user may then ascertain that the perineal retainer 100 is all the way down and seated on top of the ring tube 210. At this stage, the locking screw 180 may still be extended.

Referring to FIG. 11, to secure the perineal retainer 100 to the applicator 200, the user inserts the lock screw tool 300 into the socket or driving receptacle 186 of the screw and turn it in the first rotational direction, e.g., clockwise, all the way to a positive stop. At this stage, the perineal retainer 100 is fixed to the applicator 200. The user may ascertain that the perineal retainer 100 is attached “firmly” and that it cannot be displaced or slide along the applicator tubes 210, 215.

After securement of the perineal retainer 100 to the applicator 200 in accordance with FIG. 11, the perineal retainer 100 may be strapped to the patient such that the assembly 5 causes proper predetermined placement of the ring 205 and end portion 217 of the central tandem 215.

Because of, e.g., the curvature of the perineal retainer 100, an ergonomic and effective locating mechanism is provided. For example, referring to FIG. 3, side edges 111 have an ergonomic curved profile that facilitates placement of the perineal retainer body 110 between the patient's thighs. Further, referring to the side view of FIG. 2, the perineal retainer body has a concave shape that more comfortably and easily fits the sloping and curved profile of the patient's perineum.

It is noted that the materials of which the components of the perineal retainer 100 are formed facilitate cleaning and sterilization, among other desirable properties. In this regard, the perineal retainer body 110 is a single monolithic piece formed of PPSU (polyphenylsulfone) material, and the locking bracket 140 and the locking screw 180 are each formed, respectively, as a single monolithic piece of titanium. It should be understood however, any one or more, or all, of the three components 110, 140, 180 may be formed of different material(s) and/or be formed from multiple constituent components.

Prior to sterilization, the perineal retainer 100 may be thoroughly cleaned. Using an enzymatic detergent (e.g., Terg-A-Zyme®, MicroZyme™, or equivalent), the perineal retainer 100 may be soaked, e.g., for not less than ten (10) minutes. The perineal retainer 100 may then be transferred to an ultrasonic cleaner containing, e.g., clean enzymatic detergent. It may be desirable to not allow the components of the perineal retainer 100 to dry. The cleaner may be run for, e.g., (10) minutes. The perineal retainer 100 may then be rinsed thoroughly, e.g., three times, using sterile water, and the components of the perineal retainer 100 may be allowed to thoroughly dry before packaging for sterilization.

Sterilization of the perineal retainer 100 may be provided via an autoclave. For example, the perineal retainer 100 may be exposed to the following steam cycle:

2.1 Temperature: 132° C. (Gravity Displacement).

2.2 Exposure Dwell: 10 minutes.

2.3 Drying: 15 minutes.

Thus, the perineal retainer 100 is easily cleanable and sterilizable.

Although the present invention has been described with reference to particular examples and exemplary embodiments, it should be understood that the foregoing description is in no manner limiting. Moreover, the features described herein may be used in any combination.

What is claimed is:

1. A device for retaining a position of a treatment applicator, comprising:
a perineal retainer body configured to receive the applicator, the perineal retainer body configured to form a positive stop with a perineum of a body of a patient; and
a locking bracket configured to secure the perineal retainer body to the applicator.

2. The device according to claim 1, wherein the perineal retainer body includes curved side edges and a concave shape to conform to the body of the patient.

3. The device according to claim 1, wherein the perineal retainer body is configured to be attached to the body of the patient to prevent relative movement between the perineal retainer body and the body of the patient.

4. The device according to claim 1, wherein the perineal retainer body is attached to the body of the patient by at least one of sutures and straps.

5. The device according to claim 4, wherein the perineal retainer body includes at least one aperture configured to receive the at least one of sutures and straps.

6. The device according to claim 1, wherein the perineal retainer body includes a slot configured to receive the applicator, the slot including at least one clamping region and an enlarged region.

7. The device according to claim 6, wherein the clamping region is configured to receive and clamp a first portion of the applicator.

8. The device according to claim 6, wherein the locking bracket includes cams and the perineal retainer body includes cam followers, the cams cooperating with the cam followers to close and open the clamping region.

9. The device according to claim 8, wherein the locking bracket includes an actuator configured to move the cams relative to the cam followers, a first direction of rotation of the actuator closing the clamping region and a second, opposite direction of rotation of the actuator opening the clamping region.

10. The device according to claim 6, wherein the enlarged region is configured to movably receive a second portion of the applicator.

11. The device according to claim 1, wherein the locking bracket is movable between a first position and a second position with respect to the perineal retainer body, the locking bracket configured to exert a clamping force to clamp the applicator when the locking bracket moves from the first position to the second position, the locking bracket including an actuator configured to move the locking bracket between the first and second positions.

12. The device according to claim 11, wherein movement of the locking bracket between the first and second positions is along a line that substantially falls in a plane containing the anteroposterior axis and the dorsoventral axis of the patient when the clamped applicator is inserted into the patient.

13. The device according to claim 11, wherein the actuator is manually accessible in a direction substantially falling along a plane containing the anteroposterior axis and the dorsoventral axis of the patient when the clamped applicator is inserted into the patient.

14. The device according to claim 11, wherein the actuator is a lead screw configured to form a threaded connection with the locking bracket.

15. The device according to claim 11, wherein the locking bracket includes a camming surface configured to slide across a respective camming surface of the perineal retainer body as the locking bracket moves from the first position to the second position.

16. The device according to claim 1, wherein the device is configured to clamp two parallel tubes of the applicator.

17. The device according to claim 16, wherein the locking bracket is configured to move with respect to the perineal retainer body along an axis that is at least one of: (a) substantially coplanar with longitudinal axes of the two parallel tubes, (b) substantially parallel to a plane containing the longitudinal axes of the two parallel tubes, and (c) substantially perpendicular to the longitudinal axes of the two parallel tubes.

18. An assembly, comprising:
a treatment applicator; and
a perineal retainer device secured to the applicator, the device including:
a perineal retainer body configured to receive the applicator, the perineal retainer body configured to form a positive stop with a perineum of a body of a patient; and
a locking bracket configured to secure the perineal retainer body to the applicator.

19. A method for retaining a position of a treatment applicator, comprising:
securing a perineal retainer body to the applicator by a locking bracket; and
attaching the perineal retainer body to a body of a patient.

20. The method according to claim 19, wherein the perineal retainer body is attached to the body of the patient to prevent relative movement between the perineal retainer body and the body of the patient.

21. The method according to claim 19, wherein the perineal retainer body is attached to the body of the patient by at least one of sutures and straps.

22. The method according to claim 21, wherein the perineal retainer body includes at least one aperture configured to receive the at least one of sutures and straps.

23. The method according to claim 19, wherein the perineal retainer body is secured to the applicator by clamping the applicator in a slot of the perineal retainer body, the slot including at least one clamping region and an enlarged region.

24. The method according to claim 23, wherein a first portion of the applicator is received and clamped in the clamping region.

25. The method according to claim 23, wherein the clamping region is closed and opened by cooperation between cams on the locking bracket and cam followers on the perineal retainer body.

26. The method according to claim 25, wherein the cams are moved relative to the cam followers by an actuator engaged with the locking bracket, the clamping region being closed by a first direction of rotation of the actuator and the clamping region being opened by a second, opposite direction of rotation of the actuator.

27. The method according to claim 23, wherein a second portion of the applicator is movably received within the enlarged region.

28. The method according to claim 19, wherein the locking bracket is vertically accessible for securing and adjusting the applicator with respect to the perineal retainer body.

29. The method according to claim 19, wherein the locking bracket is moved between a first position and a second position with respect to the perineal retainer body, the locking bracket exerting a clamping force to clamp the applicator when the locking bracket moves from the first position to the second position, the locking bracket including an actuator that moves the locking bracket between the first and second positions.

30. The method according to claim 29, wherein the locking bracket moves between the first and second positions along a line that substantially falls in a plane containing the anteroposterior axis and the dorsoventral axis of the patient when the clamped applicator is inserted into the patient.

31. The method according to claim 29, wherein the actuator is manually accessible in a direction substantially falling along a plane containing the anteroposterior axis and the dorsoventral axis of the patient when the clamped applicator is inserted into the patient.

32. The method according to claim 29, wherein the locking bracket includes a camming surface that slides across a respective camming surface of the perineal retainer body as the locking bracket moves from the first position to the second position.

33. The method according to claim 29, wherein the actuator moves the locking bracket with respect to the perineal retainer body to clamp two parallel tubes of the applicator.

34. The method according to claim 33, wherein the actuator is rotatable about an axis that is at least one of: (a) substantially coplanar with longitudinal axes of the two parallel tubes, (b) substantially parallel to a plane containing the longitudinal axes of the two parallel tubes, and (c) substantially perpendicular to the longitudinal axes of the two parallel tubes.
